(19) United States
(12) Patent Application Publication
(10) Pub. No.: US 2011/0150945 A1
(43) Pub. Date: Jun. 23, 2011

(54) ORAL PHARMACEUTICAL FORMULATION FOR OMEPRAZOLE COMPRISING A SPECIFIC SEPARATION LAYER

(75) Inventor: Marco Ralf Spitz, Binzen (DE)

(73) Assignee: MEPHA GMBH, Aesch (CH)

(21) Appl. No.: 13/058,362

(22) PCT Filed: Aug. 11, 2009
(86) PCT No.: PCT/EP09/60388

§ 371 Date: Feb. 10, 2011

(30) Foreign Application Priority Data
Aug. 11, 2008 (CH) 01252/08
May 14, 2009 (CH) 00760/09

(51) Int. Cl. A61K 31/4439; A61K 9/00; A61P 1/04

(52) U.S. Cl. 424/400; 514/338

(57) ABSTRACT

The invention relates to an enteric coated oral pharmaceutical formulation comprising, as the active ingredient, omeprazole, an alkaline salt of omeprazole, an enantiomer of omeprazole, or an alkaline salt of an enantiomer of omeprazole, and a specific separation layer between active ingredient and enteric coating comprising a polyvinyl alcohol/polyethylene glycol graft copolymer and/or optionally modified pea starch.

The invention concerns an enteric coated oral pharmaceutical formulation comprising, as the active ingredient, omeprazole, an alkaline salt of omeprazole, an enantiomer of omeprazole, or an alkaline salt of an enantiomer of omeprazole, and a specific separation layer. The separation layer between active ingredient and enteric coating comprises a polyvinyl alcohol/polyethylene glycol graft copolymer and/or optionally modified pea starch.

In patent document EP 5 129 substituted 2-(pyridylmethylsulfinyl)-1H-benzimidazoles, so-called prazoles, e.g. the nowadays commercially available omeprazole, esomeprazole, pantoprazole, lansoprazole and rabeprazole, and their properties as efficient proton pump inhibitors are described. Esomeprazole is the designation for the S enantiomer of omeprazole, S-5-methoxy-2-[[(4-methoxy-3,5-dimethyl-2-pyridinyl)methyl]sulfinyl]-1H-benzimidazole. Omeprazole acts as an inhibitor of the secretion of gastric acid and is suitable for use as an anti-ulcus compound and for the prevention and treatment of diseases connected with overproduction of gastric acid in mammals and, in particular, in humans. It is known that pyridylmethylsulfinyl-benzimidazoles such as omeprazole are relatively unstable in the presence of humidity, but also in the presence of organic solvents. These compounds are particularly unstable in acid environment. A possible option for the preparation of derivatives with increased stability is the formation of alkaline salts, e.g. sodium or magnesium salts, for example as described in patent document EP 124 495 for omeprazole.

Omeprazole is usually coated with a gastric acid resistant layer, a so-called enteric coating. It has turned out to be preferable to include a separation layer between the active ingredient (in form of the free base or in form of an alkali or magnesium salt) and the enteric coating. In European patent document EP 342 522 it is proposed to coat an acid labile pyridylmethyl-sufinyl-benzimidazole first with a slightly water soluble compound such as magnesium oxide, silicium anhydride, calcium silicate, magnesium hydroxide, magnesium carbonate, aluminium hydroxide, calcium stearate or sucrose fatty acid ester, and then with a slightly water soluble film material, e.g. ethylcellulose or polyvinyl acetate. Thereafter, the standard enteric coating is applied, e.g. hydroxypropylmethylcellulose phthalate, cellulose acetate phthalate, methacrylic acid/methyl methacrylate copolymer or polyvinyl acetate phthalate. Compared to water soluble coatings the proposed separation layers provide a considerable advantage.

In EP 773 025 it is proposed to mix the acid labile pyridylmethylsufinyl-benzimidazole with hydroxypropylmethylcellulose and talcum, apply it to a core, and coat it with a separation layer made of hydroxypropylmethylcellulose and talcum, and optionally titanium oxide as a white pigment. Finally the gastric acid resistant enteric coating made from methacrylic acid/methyl methacrylate copolymer and plasticizer, e.g. triethyl citrate, is applied.

In international patent application WO 2006/085335 it is proposed to coat the acid labile active ingredient with a separation layer made from a water insoluble polymer, e.g. ethylcellulose, polyvinyl acetate, acrylic polymers and copolymers, such as Eudragit RL, Eudragit L, Eudragit RS 30D, or mixtures thereof. An organic stabilizer is added to this polymer, for example meglumine or tromethamine, or mixtures thereof, and optionally a plasticizer, e.g. polyethylene glycol, castor oil, sebacic acid dibutyl ester, triethyl citrate or mixtures thereof, and talcum in order to inhibit stickiness. Thereafter the enteric coating made from cellulose acetate phthalate, hydroxymethylcellulose, methacrylic acid/methyl methacrylate copolymer or shellac is applied.

In international patent application WO 2005/076987 pharmaceutical compositions are described, which comprise, in addition to the acid lable proton pump inhibitor of the type of prazoles, a non-steroidal anti-inflammatory active ingredient and buffer compounds. The active ingredients are packed into microcapsules to increase stability, and potential coatings are mentioned, among others also Kollicoat®, a polyvinyl alcohol/polyethylene glycol graft copolymer.

It has now surprisingly been found that remarkably stable forms of tablets, mini-tablets, pellets, film tablets, capsules, and granules of omeprazole, for example of esomeprazole, are obtained if a specific separation layer is applied between the center core comprising the active ingredient omeprazole, alkaline salt of omeprazole, enantiomer of omeprazole or alkaline salt of an enantiomer of omeprazole, and the outer enteric coating, whereby the separation layer comprises a polyvinyl alcohol/polyethylene glycol graft copolymer and/or optionally modified pea starch.

A polyvinyl alcohol/polyethylene glycol graft copolymer is available, for example, under the trade name Kollicoat® IR. Kollicoat® IR has excellent film building properties, low viscosity in a spray solution of up to 25% polymer concentration, and the layer formed on drying remains flexible, does not stick and gives an even surface without additional plasticizer.

Pea starch (optionally modified) is available, for example, under the trade name Lycoat®. Lycoat® likewise provides a flexible, non-sticky separation layer and an even surface, and can be easily sprayed on a core of active ingredient. The viscosity may be simply adapted to existing spraying equipment.

The separating layer may comprise further excipients, such as, e.g., talcum, silicic acids (Syloid®) and the like.

Both polyvinyl alcohol/polyethylene glycol graft copolymer and optionally modified pea starch may be easily applied. They provide separating layers of uniform quality, which do not influence the properties of the active ingredient, protect it from light, humidity, and acid in particular, but do not obstruct dissolution of the active ingredient in the gut.

The invention likewise comprises a method of manufacture of a solid, enteric coated oral pharmaceutical formulation comprising, as the active ingredient, omeprazole, an alkaline salt of omeprazole, an enantiomer of omeprazole, or an alkaline salt of an enantiomer of omeprazole, wherein the active ingredient, optionally with additional compounds, is applied to a core, then coated with a separation layer comprising a polyvinyl alcohol/polyethylene glycol graft copolymer and/or optionally modified pea starch, and finally coated with an enteric coating comprising cellulose acetate phthalate, hydroxymethylcellulose, methacrylic acid/methyl methacrylate copolymer, methacrylic acid/ethyl acrylate copolymer and/or shellac.

Preferred is a formulation wherein the active ingredient is esomeprazole in crystalline or amorphous form or in the form of an alkaline salt.

Particularly preferred is a formulation wherein the active ingredient is the magnesium salt of esomeprazole, in particular in the form of its hydrates, such as the dihydrate or trihydrate.

Most preferred is a formulation wherein the active ingredient is esomeprazole magnesium dihydrate.

Solid oral dosage forms considered are tablets, mini-tablets, pellets, film tablets, capsules, for example soft gelatin capsules, granules and related dosage forms.

The dosage forms consist of omeprazole, an alkaline salt of omeprazole, an enantiomer of omeprazole, or an alkaline salt of an enantiomer of omeprazole, and, optionally, the following excipients or adjuvants forming pharmaceutically acceptable oral dosage forms.

Excipients taken into consideration for solid oral dosage forms are solid powdery carriers (e.g. microcrystalline cellulose, silicium dioxide, xanthan, guar gum, magnesium aluminium silicate, calcium silicate, calcium and magnesium phosphate), aluminium oxide, titanium oxide, diluents (e.g. calcium carbonate, calcium sulfate, hydrogenated vegetable oil, kaolin, magnesium carbonate, talcum, sodium chloride), binder (e.g. guar gum, gelatin, polyvinyl-pyrrolidone, hydroxypropylmethylcellulose, hydroxyethylcellulose, hydroxypropylcellulose, traganth, alginate, carboxymethylcellulose calcium or sodium, carrageenan, xanthane), disintegrants (e.g. croscarmellose, crospovidone (crosslinked polyvinylpyrrolidone), colloidal silicium dioxide, sodium starch glycolate, sodium carboxymethyl starch, or also carrageenan), glidants (e.g. colloidal silicium dioxide, starch, tribasic calcium phosphate, talcum) and lubricants (e.g. calcium stearate, zinc stearate, magnesium stearate, stearic acid, fumaric acid, glycerol monostearate, glycerol palmitostearate, mineral oil, sodium benzoate, sodium laurylsulfate, sodium stearylfumarate, talcum, cured ricinus oil, hydrogenated castor oil).

A particularly preferred excipient is carrageenan.

Film tablets optionally comprise additional film forming compounds (e.g. carboxymethyl-cellulose sodium, carnauba wax, cellulose acetate phthalate, cetyl alcohol, gelatin, hydroxypropylmethylcellulose (HPMC), hydroxyethylcellulose (HEC), hydroxypropylcellulose (HPC), ethylcellulose, polyvinylpyrrolidone (PVP), polyvinyl alcohol (PVA), polymethacrylate, microcrystalline wax, shellac, talcum, titanium dioxide), suspension adjuvants (e.g. highly disperse silicium dioxide, kaolin, talcum), lubricants (e.g. calcium stearate, magnesium stearate, glycerol monostearate, glycerol palmitostearate, mineral oil, sodium benzoate, sodium laurylsulfate, sodium stearylfumarate, stearic acid, talcum, zinc stearate, hydrogenated castor oil), and pigments (e.g. titanium dioxide, iron oxide, pigmented indigotine, pigmented erythrosine).

Further excipients taken into consideration are polyhydroxy compounds, for example ethylene glycol, propylene glycol or butylene glycol, diethylene glycol, triethylene glycol, tetraethylene glycol and poylethylene glycol, glycerol or glycerol derivatives partially etherified with ethylene oxide. These fluid excipients are only used in such amounts that the active ingredients remain in a powdery condition.

Nitrogen containing basic organic compounds may be added, for example lysine, arginine, histidine, ethylene diamine, ethanolamine, propanolamine, N,N′-dibenzylethylene diamine, meglumine, tromethamine, choline, procaine (4-aminobenzoic acid diethylaminoethyl ester), chloroprocaine or procainamide.

Sugars are also suitable excipients for omeprazole, for example mannitol, sorbitol, dextrins, maltodextrins, inositol, isomaltol, lactitol, maltitol and xylitol, or the above mentioned aminosugar meglumine.

One or more of the above mentioned adjuvants may also be added (additionally or in place of an additive to the active ingredient) to the separation layer made from polyvinyl alcohol/polyethylene glycol graft copolymer and/or optionally modified pea starch, for example silicium oxide, titanium dioxide, talcum or a pigment. It is also possible to add a base to the separation layer, for example an inorganic base, such as sodium hydroxide, or an organic base, for example one of the organic amines mentioned above.

Capsules considered are, for example, soft gelatin, hard gelatin, HPMC, polysaccharide or starch capsules as plug capsules, welded capsules or glued capsules, of different size, colour and water content.

Granules, for example filled in sachets or bottles and the like, usually contain the above mentioned diluents, binders, disintegrants, and lubricants.

The compositions according to the invention comprise one single active ingredient selected from the group consisting of omeprazole, an alkaline salt of omeprazole, an enantiomer of omeprazole, and an alkaline salt of an enantiomer of omeprazole. Preferred active ingredient is esomeprazole or an alkaline salt of esomeprazole, for example esomeprazole magnesium, preferably as the dihydrate. All further components of the compositions of the invention contribute to the therapeutic activity of the composition, but are not active ingredients themselves.

The compositions of the invention are having two coatings, an outer gastric acid resistant enteric coating and a separation layer between the active ingredient and the enteric coating, whereby the separation layer comprises a polyvinyl alcohol/polyethylene glycol graft copolymer and/or optionally modified pea starch.

Preferred are the methods and products described in the examples.

The invention further relates to the use of compositions according to the invention for the treatment of diseases that are responsive to omeprazole, for example as anti-ulcus medicament and for the prevention and treatment of diseases connected with overproduction of gastric acid with mammals and, in particular, with humans.

Likewise the invention relates to a method of therapeutic treatment of patients in need of omeprazole, characterized in that a therapeutically effective amount of a pharmaceutical composition according to the invention is administered to the patient in need thereof.

The following examples are illustrative of the invention, but do not represent a restriction of the object of the invention.

### EXAMPLE 1

| Pos. | Material | Amount [kg] |
|---|---|---|
| 1 | Active ingredient as pellets | 150.00 |
| 2 | Kollicoat IR | 27.00 |
| 3 | Sodium hydroxide | 0.10 |
| 4 | Talcum | 11.40 |
| 5 | Silicium dioxide, highly dispersed | 8.00 |
| 6 | Titanium dioxide | 3.50 |
| 7 | Water | 340.00 |
| | Total amount of pellets after isolation | 200.00 |

Sodium hydroxide is dissolved in 90 kg water with stirring; subsequently talcum, silicium dioxide and titanium dioxide are suspended in the solution. This suspension is stirred into a solution of Kollicoat® IR in 250 kg water. The pellets with active ingredient are coated with a film using the obtained coating liquid in a fluidized bed reactor. The product temperature should be between 35 and 40° C. while coating. After coating the pellets are dried 2 hours with an air supply temperature of 60° C.

### EXAMPLE 2

| Pos. | Material | Amount [kg] |
|---|---|---|
| 1 | Active ingredient as pellets | 150.00 |
| 2 | Lycoat RS 780 (modified pea starch) | 27.00 |
| 3 | Sodium hydroxide | 0.10 |
| 4 | Talcum | 11.40 |
| 5 | Silicium dioxide, highly dispersed | 8.00 |
| 6 | Titanium dioxide | 3.50 |
| 7 | Water | 340.00 |
| | Total amount of pellets after isolation | 200.00 |

The pellets are manufactured as described in Example 1, using Lycoat® RS 780 in place of Kollicoat® IR.

### EXAMPLE 3

| Pos. | Material | Amount [kg] |
|---|---|---|
| 1 | Active ingredient as pellets | 140.00 |
| 2 | Kollicoat IR | 20.00 |
| 3 | Talcum | 10.00 |
| 4 | Titanium dioxide | 3.00 |
| 5 | Water | 280.00 |
| | Total amount of pellets after isolation | 173.00 |

Talcum and titanium dioxide are suspended in 80 kg water. This suspension is stirred into a solution of Kollicoat® IR in 200 kg water. The pellets with active ingredient are coated with a film using the obtained coating liquid in a fluidized bed reactor. The product temperature should be between 25 and 30° C. while coating. After coating the pellets are dried 2 hours with an air supply temperature of 50° C.

### EXAMPLE 4

| Pos. | Material | Amount [kg] |
|---|---|---|
| 1 | Active ingredient as pellets | 150.00 |
| 2 | Lycoat RS 780 (modified pea starch) | 27.00 |
| 3 | Talcum | 13.00 |
| 4 | Titanium dioxide | 5.00 |
| 5 | Water | 350.00 |
| | Total amount of pellets after isolation | 195.00 |

Talcum and titanium dioxide are suspended in 80 kg water. This suspension is stirred into a solution of Lycoat® RS 780 in 270 kg water. The pellets with active ingredient are coated with a film using the obtained coating liquid in a fluidized bed reactor. The product temperature should be between 35 and 40° C. while coating. After coating the pellets are dried 2 hours with an air supply temperature of 55° C.

### EXAMPLE 5

| Pos. | Material | Amount [kg] |
|---|---|---|
| 1 | Active ingredient as pellets | 150.00 |
| 2 | Kollicoat IR | 13.50 |
| 3 | Lycoat RS 780 (modified pea starch) | 13.50 |
| 3 | Talcum | 13.00 |
| 4 | Titanium dioxide | 5.00 |
| 5 | Water | 350.00 |
| | Total amount of pellets after isolation | 195.00 |

Talcum and titanium dioxide are suspended in 80 kg water. This suspension is stirred into a solution of Kollicoat® IR and Lycoat® RS 780 in 270 kg water. The pellets with active ingredient are coated with a film using the obtained coating liquid in a fluidized bed reactor. The product temperature should be between 35 and 40° C. while coating. After coating the pellets are dried 2 hours with an air supply temperature of 55° C.

### EXAMPLE 6

| Pos. | Material | Amount [kg] |
|---|---|---|
| 1 | Active ingredient as pellets | 150.00 |
| 2 | Lycoat RS 780 (modified pea starch) | 17.00 |
| 3 | Talcum | 8.00 |
| 4 | Titanium dioxide | 3.75 |
| 5 | Red iron oxide | 1.25 |
| 6 | Water | 200.00 |
| | Total amount of pellets after isolation | 180.00 |

Talcum, red iron oxide and titanium dioxide are suspended in 50 kg water. This suspension is stirred into a solution of Lycoat® RS 780 in 150 kg water. The pellets with active ingredient are coated with a film using the obtained coating liquid in a fluidized bed reactor. The product temperature should be between 35 and 40° C. while coating. After coating the pellets are dried 2 hours with an air supply temperature of 55° C.

### EXAMPLE 7

| Pos. | Material | Amount [kg] |
|---|---|---|
| 1 | Active ingredient as pellets | 120.00 |
| 2 | Kollicoat IR | 5.50 |
| 3 | Lycoat RS 780 (modified pea starch) | 5.50 |
| 4 | Talcum | 5.50 |
| 5 | Titanium dioxide | 3.50 |
| 6 | Water | 110.00 |
| | Total amount of pellets after isolation | 140.00 |

Talcum and titanium dioxide are suspended in 40 kg water. This suspension is stirred into a solution of Lycoat® RS 780 and Kollicoat® IR in 110 kg water. The pellets with active ingredient are coated with a film using the obtained coating liquid in a fluidized bed reactor. The product temperature should be between 40 and 45° C. while coating. After coating the pellets are dried 2 hours with an air supply temperature of 60° C.

### EXAMPLE 8

| Pos. | Material | Amount [kg] |
|---|---|---|
| 1 | Esomeprazole Mg dihydrate | 43.38 |
| 2 | Mannitol | 60.33 |
| 3 | Microcrystalline cellulose | 29.29 |
| 4 | Carrageenan | 7.00 |
| 5 | Sodium hydrogen carbonate | 7.00 |
| 6 | Sodium hydroxide | 3.00 |
| | Total amount of pellets | 150.00 |
| 7 | Kollicoat IR | 27.00 |
| 8 | Sodium hydroxide | 0.10 |
| 9 | Talcum | 11.40 |
| 10 | Colloidal aqueous silicium oxide | 8.00 |
| 11 | Titanium dioxide | 3.50 |
| | Total amount of pellets after isolation | 200.00 |
| 12 | Methacrylic acid-ethyl acrylate | 42.80 |
| | copolymer (1:1) (Eudragit ® L100-55) | |
| 13 | Triethyl citrate | 6.42 |
| 14 | Talcum | 21.40 |
| 15 | Titanium dioxide | 2.38 |
| | Total amount of pellets with enteric | 273.00 |
| | coating | |

Esomeprazole magnesium dihydrate is mixed with mannitol, microcrystalline cellulose and carrageenan (type Gelcarin® GP 812 NF from FMC) for 10 minutes in a positive mixer of the company Colette. Subsequently sodium hydrogen carbonate and sodium hydroxide in water are added during further 10 minutes while stirring to give an evenly moist mass. The moist mass is pressed through the 1.0 mm holes of a perforated disc with an extruder of the company Probst. The so-called extrudate is subsequently rounded with a spheronizer of the company Caleva for 4 minutes with a disc rotary speed of 1050 rpm. The obtained pellets are dried in a fluidized bed reactor of the company Aeromatik during 160 minutes with an air supply temperature of 65° C.

Sodium hydroxide is dissolved in 90 kg water while stirring. Subsequently talcum, silicium dioxide and titanium dioxide are suspended in the solution. This suspension is stirred into a solution of Kollicoat® IR in 200 kg water. The pellets with active ingredient are coated with a film using the obtained coating liquid in a fluidized bed reactor. The product temperature should be between 35 and 40° C. while coating. After coating the pellets are dried 2 hours with an air supply temperature of 60° C. Subsequently the pellets are coated with an enteric coat with standard methods.

### EXAMPLE 9

| Pos. | Material | Amount [kg] |
|---|---|---|
| 1 | Esomeprazole Mg dihydrate | 43.38 |
| 2 | Mannitol | 60.33 |
| 3 | Microcrystalline cellulose | 29.29 |
| 4 | Carrageenan | 7.00 |
| 5 | Sodium hydrogen carbonate | 7.00 |
| 6 | Sodium hydroxide | 3.00 |
| | Total amount of pellets | 150.00 |
| 7 | Kollicoat IR | 13.50 |
| 8 | Lycoat RS 780 | 13.50 |
| 9 | Sodium hydroxide | 0.10 |
| 10 | Talcum | 11.40 |
| 11 | Colloidal aqueous silicium oxide | 8.00 |
| 12 | Titanium dioxide | 3.50 |
| | Total amount of pellets after isolation | 200.00 |
| 13 | Methacrylic acid-ethyl acrylate | 42.80 |
| | copolymer (1:1) (Eudragit ® L100-55) | |
| 14 | Triethyl citrate | 6.42 |
| 15 | Talcum | 21.40 |
| 16 | Titanium dioxide | 2.38 |
| | Total amount of pellets with enteric | 273.00 |
| | coating | |

The pellets are manufactured according to Example 8 using a 1:1 mixture of Kollicoat® IR and Lycoat® RS 780 in place of Kollicoat® IR.

1. An enteric coated oral pharmaceutical formulation comprising, as the active ingredient, omeprazole, an alkaline salt of omeprazole, an enantiomer of omeprazole, or an alkaline salt of an enantiomer of omeprazole, and a separation layer between active ingredient and enteric coating comprising a polyvinyl alcohol/polyethylene glycol graft copolymer and/or optionally modified pea starch.

2. The formulation of claim 1 wherein the separation layer between active ingredient and enteric coating comprises a polyvinyl alcohol/polyethylene glycol graft copolymer.

3. The formulation of claim 1 wherein the separation layer between active ingredient and enteric coating comprises optionally modified pea starch.

4. The formulation of claim 1 wherein the separation layer between active ingredient and enteric coating comprises a polyvinyl alcohol/polyethylene glycol graft copolymer and optionally modified pea starch.

5. The formulation of claim 1 comprising omeprazole.

6. The formulation of claim 1 comprising an alkaline salt of omeprazole.

7. The formulation of claim 1 comprising esomeprazole.

8. The formulation of claim 1 comprising an alkaline salt of esomeprazole.

9. The formulation of claim 1 comprising the magnesium salt of esomeprazole.

10. The formulation of claim 1 comprising esomeprazole magnesium dihydrate.

11. The formulation of claim 1 comprising carrageenan.

12. A method of manufacture of a solid, enteric coated oral pharmaceutical formulation comprising, as the active ingredient, omeprazole, an alkaline salt of omeprazole, an enantiomer of omeprazole, or an alkaline salt of an enantiomer of omeprazole, wherein the active ingredient, optionally with additional compounds, is applied to a core, then coated with a separation layer comprising a polyvinyl alcohol/polyethylene glycol graft copolymer and/or optionally modified pea starch, and finally coated with an enteric coating comprising cellulose acetate phthalate, hydroxymethylcellulose, methacrylic acid/methyl methacrylate copolymer, methacrylic acid/ethyl acrylate copolymer and/or shellac.

13. The formulation of claim 1 as a medicament for the treatment of ulcus and for the prevention and treatment of diseases connected with overproduction of gastric acid in mammals and humans.
